(19) United States
(12) Patent Application Publication
(10) Pub. No.: US 2020/0246535 A1
(43) Pub. Date: Aug. 6, 2020

(54) SELF-FLUSHING INTRAVENOUS INFUSION SET

(71) Applicant: BOEGH INNOVATION APS, Aarhus C (DK)

(72) Inventor: Peter BOEGH JESSEN, Aarhus C (DK)

(73) Assignee: BOEGH INNOVATION APS, Aarhus C (DE)

(21) Appl. No.: 16/089,255

(22) PCT Filed: Mar. 27, 2017
(86) PCT No.: PCT/DK2017/050089

§ 371 Date: Sep. 27, 2018

(30) Foreign Application Priority Data
Mar. 29, 2016 (DK) PA 2016 70171

(51) Int. Cl. A61M 5/14; A61M 5/36

(52) U.S. Cl. CPC A61M 5/1411; A61M 2005/1403; A61M 5/1409; A61M 5/36

(57) ABSTRACT

Dual chamber drip controlling device for intravenous infusion sets enabling spontaneous flushing and a method for flushing a dual chamber drip controlling device, wherein said dual chamber comprises a flushing chamber configured as a closed compartment, said flushing chamber being tillable with flushing fluid; an administration chamber configured as a gravity based drip chamber with an inlet and outlet; and two conduits, namely conduit A and conduit B connecting the administration chamber to the flushing chamber, wherein each fluid conduit is provided with restriction means for restricting passage from the flushing chamber to the administration chamber, said restriction means being openable so as to render the administration chamber flushable.

### FIELD OF THE INVENTION

The present invention relates to a novel Intravenous infusion set. Specifically the present invention relates to an intravenous infusion set that is able of self-flushing upon emptying of the drip chamber ore administration chamber (medicine chamber) that is used for administration of the intravenous fluid, and drugs.

### BACKGROUND OF THE INVENTION

Intravenous (IV) administration of fluids and drugs is an integral part of the routine care of patients, especially those undergoing anesthesia, surgery, chemotherapy and antibiotic treatment.

The most commonly used IV infusion system consists of a bag filled with IV fluids, either prefilled with drugs ore with a possibility to add drugs before the infusion, a drip chamber, roller clamp (variable resistance controller) for control of the flow and tubing connected to an IV catheter. The elevated IV bag in this system serves as a pressure source, the roller clamp as a user-controlled resistor, and the IV catheter as a fixed resistor. Most commonly, the rate of IV fluids flow is determined by the rate at which drops of liquid are observed falling through a drip chamber.

Problems associated with existing systems include loss of expensive medicine, due to residual medicine in the system. Manuel flushing such systems is both time consuming and undesirable due to uncontrolled administration of the medicine. Thus there is uncertainty about the actual amount of medicine the patient receives (for example a patient, who receives an expensive drug may not obtain sufficient blood levels of the drug, due to the small volume of the drug, if there is residual medicine in the system). Also by manuelly flushing existing systems there is a risk of microbial infection. Further, if the liquid level is lower than the air filter there is a risk of air embolism.

It is an object of the present invention to provide an infusion system that is able of self-flushing upon emptying of either the drip chamber or administrations chamber that is used for administration of the intravenous fluid

Another object of the invention is to provide an infusion system automatically is flushed, making sure that all the drug is flushed into the patient.

Still another object of the invention is to provide an infusion system that is inexpensive to manufacture.

It is also an object of the invention to secure an accurate administration of the medicine to the patient and thereby reduce or even eliminate spread of waste medicine in to the environment.

Further, it is an object of the invention to provide a sterilized kit containing an infusion system for the administration of intravenous fluids.

### SUMMARY OF THE INVENTION

The infusion system of the present invention solves the above problems associated with prior art solutions.

In a first aspect of the present invention there is provided a dual chamber device (either localized in the administration chamber ore in the drip chamber) for IV infusions enabling spontaneous flushing. The dual chamber comprises a flushing chamber (2) configured as a closed compartment, and an administration chamber (Medicine Chamber) (3) configured as a gravity based drip chamber (or IV fluid container) with an inlet (1) and outlet (6). There are provided two conduits, namely conduit A (4) and conduit B (5) in order to connect the administration chamber (medicine chamber) (3) to the flushing chamber (2) thereby allowing fluid and/or air passage therein between. Each of the conduits (4, 5) is provided with restriction means (7, 8) for restricting passage from the flushing chamber (2) to the administration chamber (3). The restriction means (7, 8) are adapted to be either open or closed; when both are open the administration chamber (3) becomes flushable.

In a preferred embodiment the restriction means is selected from taps, valves, and membranes. Also it is preferred that the conduit A and B are in parallel alignment. Likewise it is preferred that the conduit A is located below conduit B, particularly with the central axis of conduit A in its connection point with the administration chamber being 0.1-3 mm from the central axis of conduit B in its connection point with the administration chamber. The conduits may extend into the lumen of the flushing chamber (2) and/or administration chamber (3), and the conduits may take any desired path in the lumen of the chambers provided that the conduit openings are configured to enable (automatic) flushing. Further, it is preferred that the flushing chamber and the administration chamber are parallel with their central axis and located 1-5 cm from each other. However, the flushing chamber and the administration chamber may also simply be divided by a wall of a container. Likewise the conduits A and B may be in the form of a coaxial conduits, e.g. an inner tube placed inside an outer tube.

In an alternative embodiment of the first aspect of the present invention there is provided a dual chamber device for administering a medical fluid, said dual chamber device comprising: a flushing chamber (2) configured as a closed compartment, said flushing chamber being fillable with flushing fluid; an administration chamber (3) with an outlet (6); and two conduits, namely conduit A (4) and conduit B (5) connecting the administration chamber to the flushing chamber, wherein the conduits extend into the lumen of the flushing chamber (2) and/or administration chamber (3).

In a second aspect of the present invention there is provided a method for flushing a dual (multiple) chamber drip controlling device according present invention. The method comprises the steps of opening the restriction means to allow for fluid passage in the conduits, and then allowing air to flow from the administration chamber to the flushing chamber via the conduit, while enabling flushing fluid to flow via conduit from the flushing chamber to the administration chamber.

In a third aspect of the present invention there is provided a parenteral delivery system for administering a beneficial parenteral agent formulation to a patient. The parenteral system comprises a primary reservoir comprising a parenterally administrable fluid, which is led to an administration chamber configured as a gravity based drip chamber with an inlet and outlet. This chamber is connected via two conduits to a flushing chamber, where each fluid conduit is provided with restriction means for restricting passage from the flushing chamber to the administration chamber. The restriction means are openable so as to render the administration chamber flushable as discussed above in relation to the first aspect of the present invention. Further the system includes a tube for letting fluid from the administration chamber, wherein when the parenteral delivery system is in operation, the fluid flows in the system to the patient at a parenterally therapeutically acceptable rate.

### BRIEF DESCRIPTION OF THE DRAWINGS

FIG. 1 shows a cross sectional view of the dual chamber drip controlling device of the present invention.

FIG. 2 shows a perspective view of another embodiment of the dual chamber drip controlling device of the present invention.

### DETAILED DESCRIPTION OF THE INVENTION

In the following a non-limiting embodiment of the present invention is described in more detail.

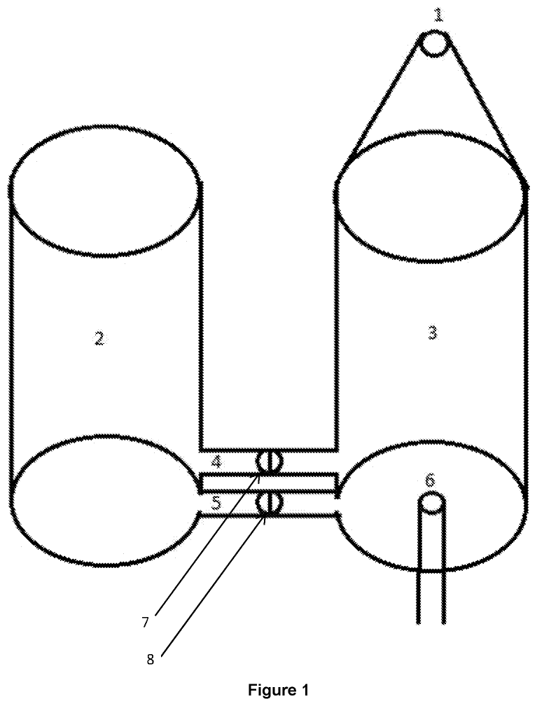

In the embodiment of FIG. 1 there is shown a means by which the dual chamber configuration enables spontaneous flushing of the chamber delivering fluid to the patient. In that embodiment there is provided a flushing chamber (2) and an administration chamber (3), the latter designed as a normal drip chamber. The administration chamber (3) is connected via two fluid conduits (4, 5) to the flushing chamber (2), which is a closed chamber. Each fluid conduit (4, 5) is provided with restrictions means (7, 8), such as a tap, membrane or valve for restricting fluid passage from the flushing chamber (2) to the administration chamber (3). These restrictions means (7, 8) may be opened when the dual chamber configuration is set to be flushable.

When the restriction means (7, 8) are opened to allow for fluid passage in the conduits (4, 5) the system is activated, and will be deployed once the liquid level in the administration chamber (3) is lower than the upper fluid conduit (4) thereby allowing air to flow from the administration chamber (3) to the flushing chamber (2) via the upper conduit (4), while enabling flushing fluid to flow via the lower conduit (5) from the flushing chamber (2) to the administration chamber (3). Accordingly, the contents of the administration chamber (e.g. drug fluid) will be emptied.

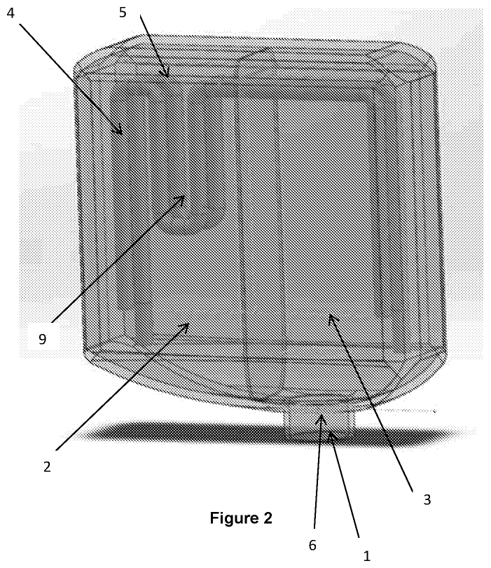

Referring to FIG. 2 there is shown an embodiment in which the administration and flushing chambers are merely divided by a wall in a container. In this embodiment another way of restricting the flow through the conduits has been realized by increasing the length of the conduits, and thereby decreasing the diffusion rate. Moreover, as shown in FIG. 2 a loop (9) may be installed in order to further restrict the flow and particularly the diffusion of ingredients (e.g. medicine) from the administration chamber toward the flushing chamber. It should be noted that in the embodiment shown in FIG. 2 the inlet and outlet are in the approximately the same place, however, this configuration could be changed by a skilled person without departing from the basic concept of the present invention.

As will be understood by a skilled person the geometrical shape of the administration chamber may be configured to minimize the residual volume to be flushed; e.g. by providing a cone-shaped chamber with the tip pointing down. Moreover, an air-guard device or filter could be implemented.

1. A dual chamber drip controlling device for IV infusion sets enabling spontaneous flushing, said dual chamber comprising:
a flushing chamber configured as a closed compartment, said flushing chamber being fillable with flushing fluid;
an administration chamber configured as a gravity based drip chamber with an inlet and outlet; and
two conduits, namely conduit A and conduit B connecting the administration chamber to the flushing chamber;
wherein each fluid conduit is provided with restriction means for restricting passage from the flushing chamber to the administration chamber, said restriction means being openable so as to render the administration chamber flushable.

2. The dual chamber drip controlling device according to claim 1, wherein the restriction means is selected from taps, valves, and membranes.

3. The dual chamber drip controlling device according to claim 1, wherein conduits A and B are in parallel alignment or are extended so as to increase the diffusion through them.

4. The dual chamber drip controlling device according to any claim 1, wherein conduit A is located above conduit B.

5. The dual chamber drip controlling device according to claim 1, wherein the central axis of conduit B in its connection point with the administration chamber is 0.1-3 mm from the central axis of conduit A in its connection point with the administration chamber.

6. The dual chamber drip controlling device according to claim 1, wherein the flushing chamber and the administration chamber are parallel with their central axis located 1-5 cm from each other.

7. The dual chamber drip controlling device according to claim 1, wherein the flushing chamber contains flushing fluid constituting 5-90%, preferably 10-80%, more preferably 15-65%, and most preferably 18-60% of the volume of the flushing chamber.

8. The dual chamber drip controlling device according to claim 1, wherein the flushing chamber is provided with opening means for filling it with flushing fluid.

9. The dual chamber drip controlling device according to claim 1, wherein the conduits extend into the lumen of the flushing chamber and/or administration chamber.

10. A method for flushing a dual (multiple) chamber drip controlling device according to claim 1, said method comprising the steps:
opening the restriction means to allow for fluid passage in the conduits;
allowing air to flow from the administration chamber to the flushing chamber via the upper conduit, while enabling flushing fluid to flow via lower conduit from the flushing chamber to the administration chamber.

11. The dual chamber device for administering a medical fluid, said dual chamber device comprising: a flushing chamber configured as a closed compartment, said flushing chamber being fillable with flushing fluid;
an administration chamber with an outlet; and two conduits, namely conduit A and conduit B connecting the administration chamber to the flushing chamber, wherein the conduits extend into the lumen of the flushing chamber and/or administration chamber.

12. The dual chamber device of claim 11, wherein each fluid conduit is provided with restriction means for restricting passage from the flushing chamber to the administration chamber.

13. A parenteral delivery system for administering a beneficial parenteral agent formulation to a patient, the parenteral system comprising:
a primary reservoir comprising a parenterally administrable fluid;
a flushing chamber containing flushing fluid configured as a closed compartment;
an administration chamber configured as a gravity based drip chamber with an inlet and outlet; and
two conduits connecting the administration chamber to the flushing chamber;
wherein each fluid conduit is provided with restriction means for restricting passage from the flushing chamber to the administration chamber, said restriction means being openable so as to render the administration chamber flushable;
a tube for letting fluid from the administration chamber;
wherein when the parenteral delivery system is in operation, the fluid flows in the system to the patient at a parenterally therapeutically acceptable rate.
